(19) United States
(12) Patent Application Publication
(10) Pub. No.: US 2004/0220105 A1
(43) Pub. Date: Nov. 4, 2004

(54) Protracted GLP-1 compositions

(76) Inventors: Ejvind Jensen, Birkerod (DK); Klavs Jorgensen, Virum (DK)

Correspondence Address: NOVO NORDISK PHARMACEUTICALS, INC, 100 COLLEGE ROAD WEST, PRINCETON, NJ, 08540, US

(21) Appl. No.: 10/854,620

(22) Filed: May 26, 2004

Related U.S. Application Data
(63) Continuation of application No. 09/767,981, filed on Jan. 23, 2001.
Application No. 09/767,981 is a continuation of application No. 08/860,103, filed on Jun. 17, 1997, now abandoned.
Application No. 08/860,103 is a 371 of international application No. PCT/DK95/00516, filed on Dec. 21, 1995.

(30) Foreign Application Priority Data
Dec. 23, 1994 (DK) 1478/94

(51) Int. Cl. A61K 38/26

(52) U.S. Cl. 514/12

(57) ABSTRACT

Thixotropic compositions containing GLP-1 compounds have protracted action.

### FIELD OF THIS INVENTION

The present invention relates to a composition containing GLP-1 compounds having protracted action and to a process for preparation thereof

### BACKGROUND OF THIS INVENTION

Diabetes is characterized by an impaired glucose metabolism manifesting itself among other things by an elevated blood glucose level in the diabetic patients. Underlying defects lead to a classification of diabetes into two major groups, i.e., type I and type II diabetes. Type I diabetes, also designated insulin demanding diabetes mellitus (IDDM), arises when patients lack β-cells producing insulin in their pancreatic glands. Type II diabetes, also designated non-insulin dependent diabetes mellitus (NIDDM), occurs in patients with an impaired β-cell function besides a range of other abnormalities.

Type I diabetic patients are currently treated with insulin while the majority of type II diabetic patients are treated either with agents that stimulate β-cell function or with agents that enhance the tissue sensitivity of the patients towards insulin.

Glucagon-like peptide-1, also designated GLP-1, is a peptide sequence found as a constituent of mammalian proglucagon. In 1985, it was demonstrated that GLP-1(1-36) amide stimulates insulin release from isolated precultured rat pancreatic islets in the presence of glucose in a dose-dependent manner. This finding suggests that GLP-1(1-36) amide and related peptides might be useful in the treatment of type II diabetes. In recent years, particular interest has focused on GLP-1 fragments such as GLP-1(7-37) and GLP-1(7-36) amide and analogues and functional derivatives thereof. Hereinafter, these compounds are designated GLP-1 compounds.

It has been found that GLP-1 compounds such as GLP-1(7-37) and GLP-1(7-36) amide have a too fast action when administered to human subjects. Therefore, there is a need for compositions containing GLP-1 compounds and having a protracted action. The availability of such protracted compositions will spare the diabetics the chore and discomfort of multiple daily injections.

Apparently, some theoretical possibilities of controlling the duration of action of GLP-1(7-37) is described at the bottom of Column 6 in U.S. Pat. No. 5,120,712. The possibilities mentioned therein are the use of polymers to complex or adsorb GLP-1(7-37), the selection of appropriate macromolecules (for example, protamine sulphate is mentioned among other), the incorporation of GLP-1(7-37) into particles of a polymeric material or the entrapment of GLP-1(7-37) in microcapsules.

A huge number of possible ways of preparing prolonged delivery of certain GLP-1 compounds is desribed in a European patent application having publication number 619,322. The possibilites mentioned therein are to add a polymer, to prepare an oil suspension, to add zinc (II), to add a metal, to add a basic polypeptide, to add a phenolic compound, to prepare an amorphous/crystalline formulation, or to use a liposome delivery system.

None of these known compositions are gels or thixotropic compositions.

One object of this invention is to provide compositions containing GLP-1 compounds and having a protracted action.

A further object of this invention is to provide compositions containing GLP-1 compounds and having a sufficient high stability, e.g. chemical stability and, especially, physical stability.

### BRIEF DESCRIPTION OF THIS INVENTION

Surprisingly, it has been found that compositions containing a GLP-1 compound and a phenolic and/or an alcoholic aromatic compound in certain concentrations result in a thixotropic gel showing a protracted release of the active GLP-1 compound.

### DETAILED DESCRIPTION OF THIS INVENTION

This invention deals with compounds having GLP-1 like activity herein referred to as GLP-1 compounds. GLP-1 compounds bind to the GLP-1 receptor (vide Proc.Nat. Acad.Sci.USA 89 (1992), 8641). Examples of specific GLP-1 compounds are polypeptides comprising the 7-34 amino acid sequence of GLP-1, viz. formula I:

His-Ala-Glu-Gly-Thr-Phe-Thr-Ser-Asp-Val-Ser-Ser-Tyr-Leu-Glu-Gly-Gln-Ala-Ala-Lys-Glu-Phe-Ile-Ala-Trp-Leu-Val-Lys (I)

or a peptide sequence derived from formula I without eliminating the GLP-1 like activity. The term GLP-1 compound also comprises derivatives of said polypeptides such as acid addition salts, carboxylate salts, lower alkyl esters, amides, lower alkyl amides and lower dialkyl amides.

The compositions of this invention are gels. In a preferred embodiment, the gels have thixotropic properties. One way of preparing the thixotropic gels according to this invention is to mix the GLP-1 compound with a phenolic or an alcoholic aromatic compound in an aqueous medium. Preferably, the phenolic or alcoholic aromatic compound is a pharmaceutically acceptable antimicrobial preservative. Non-limiting examples of such compounds include benzyl alcohol, a cresol, e.g., m-cresol, a phenol, e.g., phenol or resorcinol, or a paraben, e.g., methyl paraben or propyl paraben. The compositions of this invention may contain both a phenolic or an alcoholic aromatic compound and a divalent metal ion, preferably in the form of a salt. A preferred ion is Zn(II). Other metal ions may also be used including Ca(II), Mg(II), Co(II), Mn(II), Fe(II), and Cu(II). For example, the divalent metal salts can be the chloride or another pharmaceutically acceptable salt. Depending on which process has been used during the purification of the GLP-1 compound, it is, in some cases preferred to add an acetate and, in other cases, preferred to avoid the presence of acetate in the final GLP-1 composition.

The compositions of this invention can be prepared by using the GLP-1 compound in a concentration within a certain range. Consequently, a preferred embodiment of this invention is compositions containing not less than about 2 mg/ml, preferably not less than about 5 mg/ml, more preferred not less than about 10 mg/ml of a GLP-1 compound and, preferably, containing not more than about 100 mg/ml of a GLP-1 compound.

Another preferred embodiment of this invention relates to a thixotropic composition containing no compounds which are known to form thixotropic mixtures. It is novel that GLP-1 compounds and phenolic or alcoholic aromatic compounds which can safely be administered to human beings in medicaments can form thixotropic gels.

In addition to the specific ingredients which are to be present in the compositions of this invention, said composition may also, in addition to water, contain a pH buffering agent, an osmotic pressure controlling agent or other ancillary agents.

The compositions of this invention can be used as an insulinotropic agent in the treatment of diabetes. The dosage to be administered to human subjects is conveniently determined by a physician. The dosage may be in the range 1-1,000 μg/kg/day. Normally, the compositions of this invention are administered subcutaneously or intramuscularly.

The features disclosed in the present description, examples and claims may, both separately and in any combination thereof, be material for realizing this invention in diverse forms thereof.

This invention is further illustrated by the following examples which are not to be construed as limiting, but merely as an illustration of some preferred features of this invention. Additional preferred embodiments of this invention are stated in the claims.

Study of gelling and thixotropic properties

When evaluating compositions for their gelling and thixotropic properties, the following tests, which are performed at a temperature of 20-25° C., can be used:

Step 1: A cylindrical glass vial having a flat bottom, an inner diameter of about 6.4 mm and a height of about 4 cm is filled to a height of about 1 cm from the bottom with the composition which is to be tested. The filling can be made using a syringe. The glass vial used can be a 1 ml Clear Glass Vial With Caps from Waters, USA (part No. 78514).

Step 2: The vial is equipped with a cap and is stored for 24 hours.

Step 3: After removal of the cap, a glass ball is very cautiously placed at the top of the composition to be tested. This glass ball has a weight of about 17-18 mg and a diameter of about 2.4 mm. After standing for 1 hour, the glass ball should not sink more than about 5 mm.

Step 4: Thereafter, the vial is placed in a vortex mixer (for example, a whirlimixer from Fisons Scientific Apparatus, England) and shaken. During this mixing step, the ball shall drop to the bottom.

For preferred compositions according to this invention the glass ball should not sink more than about 5 mm after standing for 5 hours in step 3 above, and more preferred it does not sink more than about 5 mm after standing for 24 hours in step 3 above.

A still further feature of preferred compositions according to this invention is that Step 4 is followed by the following steps:

Step 5: The vial is equipped with a cap and is stored for 24 hours.

Step 6: Thereafter, the vial is turned upside-down.

Step 7: After standing for 1 hour, the glass ball should not sink more than about 5 mm.

For preferred compositions according to this invention the glass ball should not sink more than about 5 mm after standing for 5 hours in step 7 above, and more preferred it does not sink more than about 5 mm after standing for 24 hours in step 7 above.

Absorption studies

The absorption of the GLP-1(7-37) compositions, described in the examples, were studied in pigs after subcutaneous injection. The compositions were made from a mixture of GLP-1(7-37) and a trace amount of ¹²⁵I-GLP-1(7-37). One composition was injected at one side of the neck and another composition at the other side in each of 6 pigs. The absorption was followed by external monitoring of the radioactivity remaining at the site of injection. The injections were performed by NovoPen® to a depth of 5 mm.

### EXAMPLE 1

The zinc free gel composition of this invention designated A was: 20 mg/ml GLP-1(7-37), 16 mg/ml glycerol, and 3 mg/ml m-cresol (pH value: 7.2).

This composition was made by mixing 2.5 ml acidic GLP-1(7-37) solution (20 mg/ml) with 7.5 μl of m-cresol and 40 mg of glycerol, followed by adjustment of the pH value, which was made possible by the thixotropic properties of the gel that assumed low viscosity by stirring. A high viscosity gel was formed soon after stirring was stopped. 60 μl was injected in each pig.

### EXAMPLE 2

The zinc containing gel composition of this invention designated B was: 20 mg/ml GLP-1(7-37), 0.5 mmol/l Zn⁺⁺, 16 mg/ml glycerol, and 3 mg/ml m-cresol. The molar ratio between Zn⁺⁺ and GLP-1(7-37) was 0.08.

This composition was made by mixing 1 ml of GLP-1(7-37) solution (40 mg/ml), adjusted to a pH value of 7.4, with 1 ml of a solution containing 6 g/l m-cresol, 32 g/l glycerol and 1 mmol/l zinc acetate. A high viscosity gel was formed soon after mixing. 80 μl were injected in each pig.

### EXAMPLE 3

The zinc containing gel composition of this invention designated C was: 20 mg/ml GLP-1(7-37), 1 mmol/l Zn⁺⁺, 16 mg/ml glycerol, and 3 mg/ml m-cresol. The molar ratio between Zn⁺⁺ and GLP-1(7-37) was 0.17.

This composition was made by mixing 1 ml of GLP-1(7-37) solution (40 mg/ml), adjusted to a pH value of 7.4, with 1 ml of a solution containing 6 g/l m-cresol, 32 g/l glycerol and 2 mmol/l zinc acetate. A high viscosity gel was formed soon after mixing. 80 μl were injected in each pig.

### EXAMPLE 4

The zinc containing gel composition of this invention designated D was: 20 mg/ml GLP-1 (7-37), 2 mmol/l Zn⁺⁺, 16 mg/ml glycerol, and 3 mg/ml m-cresol. The molar ratio between Zn⁺⁺ and GLP-1(7-37) was 0.33.

This composition was made by mixing 1 ml of GLP-1(7-37) solution (40 mg/ml), adjusted to a pH value of 8.7, with a 1 ml of a solution containing 6 mg/ml m-cresol, 32 g/l glycerol, and 4 mmol/l zinc acetate. A high viscosity gel was formed soon after mixing. 80 μl were injected in each pig.

### EXAMPLE 5

As a non-gel, non-protracted solution of GLP-1(7-37) for use as a reference in the absorption studies, the following low concentrated zinc free GLP-1(7-37) composition designated REF was chosen: 1 mg/ml GLP-1(7-37), 16 mg/ml glycerol, 3 mg/ml phenol (pH value: 7.3).

The results of the absorption studies from Examples 1-5 are shown in the Table below.

TABLE

| Time after | % Residual radioactivity | | | | |
|---|---|---|---|---|---|
| injection | Prepara- | Prepara- | Prepara- | Prepara- | Prepara- |
| Hours | tion A | tion B | tion C | tion D | tion REF |
| 0 | 100 | 100 | 100 | 100 | 100 |
| 1 | — | — | — | — | 42.6 |
| 1.5 | 70.6 | — | — | — | — |
| 2 | — | 64.1 | 76.6 | 94.7 | — |
| 3 | 36.8 | — | — | — | 4.5 |
| 4 | — | 44.4 | 67.0 | 91.3 | — |
| 5 | 10.3 | — | — | — | 1.9 |
| 6 | — | 32.7 | 60.8 | — | — |
| 6.5 | — | — | — | — | 1.7 |
| 7 | 3.4 | — | — | — | — |
| 15.5 | — | — | — | 59.2 | — |
| 21.5 | — | — | — | 40.3 | — |
| 24 | — | 2.4 | 12.8 | — | 1.1 |
| 40 | — | — | — | 9.1 | — |
| T-50% | 2.3 | 3.3 | 8.4 | 19.3 | 0.8 |
| (hours)* | | | | | |

*Time until 50% of initial radioactivity remaining in the tissue, calculated on the basis of exponential disappearance between adjacent time points.

As appears from these data, the compositions of this invention are considerably more protracted than the reference solution.

1. A composition containing a GLP-1 compound which composition is a gel.

2. A composition, according to claim 1, which composition has thixotropic properties.

3. Composition, according to claim 1 or 2, containing not less than about 2 mg/ml, preferably not less than about 5 mg/ml, more preferred not less than about 10 mg/ml of a GLP-1 compound and, preferably, containing not more than about 100 mg/ml of a GLP-1 compound.

4. Composition, according to any one of the preceding claims, containing a phenolic or an alcoholic aromatic compound.

5. Composition, according to the preceding claim, wherein the phenolic or alcoholic aromatic compound is a pharmaceutically acceptable antimicrobial preservative.

6. Composition, according to the preceding claim, wherein the pharmaceutically acceptable antimicrobial preservative is benzyl alcohol, a cresol, e.g., m-cresol, a phenol, e.g., phenol or resorcinol, or a paraben, e.g., methyl paraben or propyl paraben.

7. Composition, according to any one of the preceding claims, wherein the thixotropic property only or mainly results from the presence of a GLP-1 compound.

8. Composition, according to anyone of the preceding claims, wherein the thixotropic property only or mainly results from the presence of a GLP-1 compound together with a pharmaceutically acceptable antimicrobial preservative.

9. Composition, according to anyone of the preceding claims, containing divalent metal ions, e.g. zinc, calcium, magnesium or cobalto ions.

10. Composition, according to the preceding claim, wherein the metal ions are zinc ions.

11. Composition, according to anyone of the preceding claims, containing 1 zinc ion per molecule of the GLP-1 compound or less and, preferably, they contain less than 0.4 zinc ion per molecule of the GLP-1 compound, more preferred they contain between 0.4 and 0.1 zinc ion per molecule of the GLP-1 compound and most preferred between 0.2 and above 0.1 zinc ion per molecule of the GLP-1 compound.

12. A method for the treatment of diabetes mellitus in a mammal in need of such treatment comprising the administration of a composition according to any one of the preceding claims containing an effective amount of the GLP-1 compound.

13. A method, according to the preceding claim, wherein the administration is performed by subcutaneous injection.

14. Any novel feature or combination of features described herein.
